(19) United States
(12) Patent Application Publication
(10) Pub. No.: US 2010/0256215 A1
(43) Pub. Date: Oct. 7, 2010

(54) Menthol-Melatonin Dissolving Film

(75) Inventors: Hanford N. Lockwood, JR., San Mateo, CA (US); Michele Lockwood, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Silver Eagle Labs NV, LLC, Carson City, NV (US)

(21) Appl. No.: 12/417,551

(22) Filed: Apr. 2, 2009

(51) Int. Cl. A61K 31/4045

(52) U.S. Cl. 514/415

(57) ABSTRACT

A fast-dissolving film to be placed in the oral cavity of a person includes melatonin, or melatonin analog, with or without menthol, or menthol analog, that can be absorbed through the oral mucosa directly into the person's bloodstream. The film is thin and made from a low-moisture, non-tacky material that becomes dissolved when hydrated and that is sized so that the substrate can be placed in oral cavities of persons. From 0.3 mg to 10 mg melatonin, or a melatonin analog, and, optionally, menthol, or a menthol analog, are added to the substrate, which is then packaged in a flat, sealed container that can be unobtrusively placed into a pocket carried by the person, thereby making the container readily accessible to the person. In use, the film is removed from the container and placed into the person's oral cavity so that it contacts a mucosal membrane in the oral cavity of the person to thereby hydrate the substrate. The melatonin is thereby liberated from the film so that it can enter the person's bloodstream via the mucosal membrane.

### BACKGROUND OF THE INVENTION

This invention relates to the oral mucosal delivery of menthol and melatonin or their analogs utilizing existing dissolving film technologies and to a product that delays the onset of snoring and acts as a natural aid in the treatment of age-related sleeplessness, jet lag, or other disorders related to sleep.

Menthol is an organic compound made synthetically or obtained from peppermint or other mint oils. It is a pungent, waxy, crystalline substance, clear or white in color. Menthol has local anesthetic and counterirritant qualities, as well as local vasodilation characteristics. Because of its ability to rapidly penetrate the mucosal membranes in the nose and throat, menthol is widely used as a decongestant, as well as a remedy for minor sore throats. The local vasodilation characteristic of menthol has been shown to be efficacious in the rapid delivery of ibuprofen, nitroglycerin, and other pharmacologically active ingredients. Clinical studies have shown that menthol can dramatically increase the transdermal delivery of medications and/or herbal supplements.

Melatonin is a naturally occurring hormone found in most animals, including humans, which is important in the regulation of the circadian rhythms of several biological functions, especially the sleep cycle. Some of the biological effects of melatonin are produced through activation of melatonin receptors, while others are due to its role as a pervasive and powerful antioxidant with a particular role in the protection of nuclear and mitochondrial DNA. Exogenous melatonin taken in the evening is the standard treatment for delayed sleep phase syndrome. It also appears to be effective in the relief of jet lag and the sleep problems encountered by people who work night shifts. If taken in pill form 30 to 90 minutes before bedtime, melatonin enters the bloodstream earlier than the brain's own melatonin production and acts as a mild hypnotic.

Research studies have also observed that melatonin may be useful in the treatment of Alzheimer's disease, reduces the time needed to fall asleep for patients suffering from attention-deficit/hyperactivity disorder, improves the thyroid function and gonadotropin levels in perimenopausal women, is an effective treatment for migraines, cluster headaches and seasonal affective disorder, may have potential use in the treatment of some cancers, and may be useful in treating obesity. The hormone is available as oral supplements (capsules, tablets or liquid) and as transdermal patches.

The methods for delivering medicines or drugs into the human system through the skin, nasal passages, eyes or mouth in the prior art reveal fast-dissolving oral films for breath fresheners, chewing gum or lozenges, beverages, nasal sprays, eye drops, and gel or ointment compositions applied to the skin either directly or as dermal patches. The mucosal or percutaneous absorption of drugs has the advantages of avoiding metabolization of the drugs in the liver, of rapid delivery, and of the lowering of dosages of the administered drugs.

The product of the present invention is a pleasant tasting, rapidly dissolving film which contains a low dosage of melatonin or melatonin analog in combination with menthol or menthol analog. The film is placed on the back of the tongue or roof of the mouth. As the film dissolves, the ingredients coat the tissues of the throat, which is essential in the mitigation of snoring. Within seconds the film dissolves, and melatonin is rapidly absorbed into the bloodstream through the mucosal membranes of the mouth with the aid of menthol. Within a few seconds of entering the bloodstream, melatonin reaches the brain. For users who have trouble sleeping, this invention provides relief sooner than that experienced with melatonin pills digested in the intestinal system. The user can also regulate the quantity of melatonin by consuming a successive number of anti-snore dissolving films according to his own needs. If the user awakens during the night, he can safely take another film without fear of encountering “drug hangover” the next morning.

U.S. Pat. No. 7,476,405 by Gardiner et al. teaches a method for orally administering a composition comprising melatonin and herbal pain relievers to improve the quality of a person's sleep and to alleviate minor aches and pains. This composition is delivered via capsule, tablet, caplet, a liquid beverage, a powder beverage mix, a dietary gel, or preferably a timed release caplet.

U.S. Pat. Nos. 7,407,669 and 7,025,983 by Leung et al. disclose a fast-dissolving orally consumable film used to deliver breath deodorizing agents, antimicrobial agents and salivary stimulants to the oral cavity. The films can also be used to deliver pharmaceutically active agents. Leung discloses an improvement to films in the prior art, as well as to processes for making them.

U.S. Pat. No. 6,998,112 by Zuckerman discloses a toothpaste composition containing melatonin to induce sleep while promoting intraoral cleanliness. The composition can be in the form of a dental cream or mouth spray.

U.S. Pat. No. 6,559,180 by Busiashvili reveals the effect of menthol on the dosage of nitroglycerin. When used in combination with menthol, the dosage of nitroglycerin can be lowered without sacrificing effectiveness, but reducing the common side effects of taking nitroglycerin such as headache and fainting.

This invention is an improvement over the prior art in that it delivers melatonin or melatonin analog in a rapidly dissolving film with menthol or menthol analog to aid in the early onset of sleep and relief from snoring.

### SUMMARY OF THE INVENTION

It is an object of the present invention to provide a mechanism that delivers a small amount of melatonin and menthol or their analogs through the oral mucosa in the user's mouth.

The present invention utilizes the unique features of consumable films that are low in moisture, non-tacky in texture and substance, and rapidly dissolvable by composition. Melatonin or a melatonin analog from 0.3 mg to 10 mg and menthol or menthol analog from 5% to 10% by weight is mixed with, sprayed on, injected into or otherwise applied to the film before the drying process. Rolls of melatonin-injected film containing menthol are then stored in protective wrappers until cut by machine and packaged in individual foil wrappers or bundled in cassettes for commercial use. Both the rolls of melatonin-injected film containing menthol and the individually foil-wrapped dissolving strips with melatonin and menthol can be stored at room temperature.

A further advantage of the present invention is that it requires no special handling or storage. The preferred individually foil-wrapped packaging of the present invention provides stability and freshness to the dissolving film delivery system containing melatonin and menthol or their analogs. A side benefit of the pleasant-tasting film is the improvement in malodorous breath.

### BRIEF DESCRIPTION OF THE DRAWINGS

FIG. 1 is a perspective, front elevational view of a flat container for sealingly storing the melatonin-containing film which has a peel-back sheet to provide access to the film; and

FIG. 2 is a side elevational view, in section, of a container for holding several melatonin-bearing films constructed in accordance with the present invention.

### DESCRIPTION OF THE PREFERRED EMBODIMENTS

The present invention is a unique system for providing users with relief from insomnia and other sleep-related disorders. The menthol-melatonin strip is an invention that utilizes dissolving film technology as a delivery system for menthol and melatonin or their analogs to relieve the user's insomnia and snoring.

The film rapidly dissolves on the user's tongue or the roof of the mouth allowing melatonin to be quickly absorbed through the mucosal membranes into the bloodstream with the aid of menthol offering rapid relief to the non-sleeper or snorer.

The current invention is a combination of menthol and melatonin or their analogs with dissolving film technology which rapidly delivers the ingredients to the user. The dissolving film is low in moisture, non-tacky in texture, and designed to dissolve rapidly when in contact with the moisture in a user's mouth. The dissolving film cut during the manufacturing process is a strip between 1.5 to 3 mils thick and square or rectangular in shape with a surface area from 0.5 to 2 square inches. The size of the preferred embodiment is a strip 0.8 inches wide by 1.25 inches long with a weight of 55 mg. In a preferred embodiment of the present invention, the melatonin content of the strip is between about 0.3 to 10 mg and the menthol content of the strip is between about 5% to 10% by weight, depending on the use of the product.

The dissolving film is made through a process where the ingredients are thoroughly mixed in an aqueous solution that contains both the constituents of the dissolving film and the active ingredients, which in this case are menthol and melatonin or their analogs. The aqueous solution is then dried at the appropriate temperature and time interval to create the desired film thickness and ingredient composition. The dried film is cut to the dimensions of the desired strip size containing the appropriate dosage of the active ingredients.

In the preferred embodiment, the fast-dissolving film can contain hydroxypropyl methylcellulose, microcrystalline cellulose, carrageenan, alpha bisabolol, polysorbate 80, glycerin, artificial sweeteners, flavoring or food coloring, combined with the active ingredients of melatonin and menthol. The hydroxypropyl methylcellulose, microcrystalline cellulose, carrageenan, alpha bisabolol, polysorbate 80, and glycerin make up the base substrate of the film. This substrate is vegetarian-based allowing the widest possible use by the population. In addition the use of artificial sweeteners, such as sucralose, allows diabetic persons to use this invention safely. The flavoring of the melatonin and menthol film is designed to disguise or mask the taste of ingredients. In the preferred embodiment, a wintergreen flavoring is used; however, any other strong mint or natural flavoring can be used to cover up the taste of the ingredients. Although the preferred embodiment of this invention is vegetarian-based, it is possible to use an animal product-based substrate with an artificial sweetener, appropriate flavoring and food coloring with the menthol and melatonin in the manufacture of the film.

The processed film, or menthol-melatonin strip, is hydroscopic and can experience off-gassing of ingredients which can degrade the product over time if it is not packaged properly. One packaging option is the use of plastic cassettes which can hold up to 24 strips. The cassette in this application would be slightly larger than the strips to minimize the air volume in the cassette. The cassette is then packaged in a blister pack with plastic on one side and a foil seal between the cassette and the cardboard backing of the blister pack. This packaging is airtight until the cassette is removed from the blister pack. Once the cassette is removed from the blister pack and the first strip is removed, however, the remaining strips must be consumed in a short period of time before the strips absorb too much moisture and degrade, unless the strips are individually sealed as well. There is also the risk of removing two or more strips from a stack of strips as they begin to stick together, causing the user to ingest a greater dose of menthol and melatonin than desired or expected.

The preferred packaging option is to place an individual menthol-melatonin strip in its own airtight foil wrapper. This packaging gives each strip an indefinite shelf life and ensures that the user will always consume the correct amount of ingredients. The individual foil-wrapped menthol-melatonin strips can then be packaged in a box or in a conventional blister pack container.

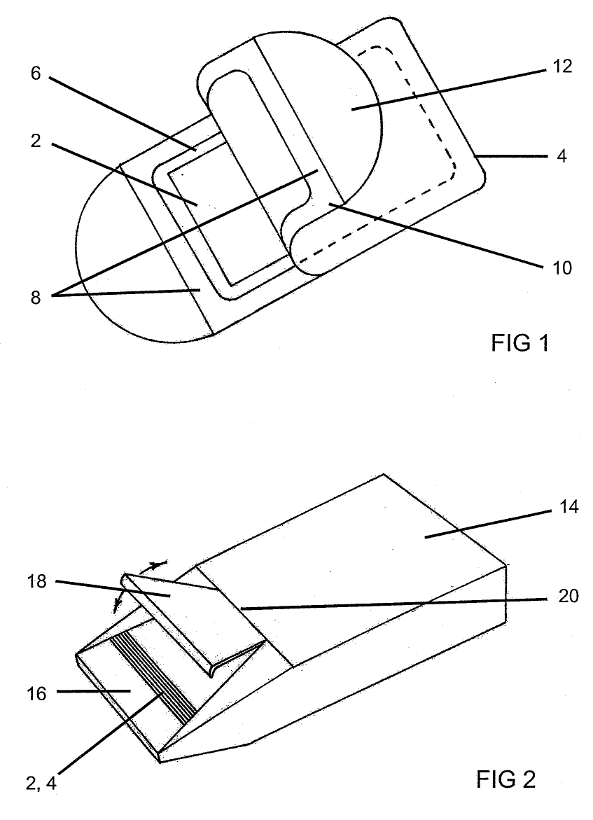

Referring to FIG. 1, in a presently preferred embodiment a melatonin-carrying film 2 constructed in accordance with the present invention as above described has a rectangular outline and is thin (preferably in the range between about 1.5 to 3 mils) so that it is flexible and can be readily placed in a person's oral cavity. The film is placed inside a container constructed of a gas-impervious material, such as, for example, any one of a large variety of plastics or foil used for packaging, and has a depth (not separately shown in FIG. 1) sufficient to readily accommodate one film. A top 6 of the container is open, has outwardly extending flanges 8 that surround the film inside the container, and includes a peel-back sheet or membrane 10 that is sealingly secured, e.g. bonded, to the lateral flanges 8 of the container. The bond of the flexible membrane keeps the interior of the container sealed from the exterior, thereby preventing a degassing of components of the film and/or its contamination, while the bond is sufficiently weak so that the membrane is readily pulled back to expose the film for removal from the container. To facilitate the peel-back of the membrane, it may include a tab 12 where the membrane can be grasped for pulling it off the flanges of the container. The thickness of the container is kept thin to facilitate carrying it in pockets and the like. Its thickness can be as small as 1 to 3 mm to accommodate the thickness of the film plus the thickness of the container walls on top of and below the strip in the container.

In use, the person places one or more of the sealed containers 4 into his pocket, for example, and carries the strips with him at all times so that the strip is available when needed. When the user wishes to take a melatonin strip, for example to more easily fall asleep or to prevent snoring, he places his hand into the pocket or briefcase, peels back membrane 10 from container 4, and then slides the film out of the opened container with his fingers. By slightly moistening his fingers prior to touching the film, the film will adhere to the user's finger, which facilitates placing the film in his oral cavity.

To provide an extra supply of melatonin-carrying films which, for example, is sufficient to provide a supply of strips over some period of time, for example for a week or more, a larger number of melatonin-carrying films can be placed into a multi-film holding cartridge 14 as illustrated in FIG. 2. One end 16 of the cartridge is open, and a cover 18 is provided for closing the open cartridge end. A pivot 20 can be provided to secure the cover to the cartridge to facilitate closing of the cartridge after use.

As previously mentioned, a number of melatonin-bearing films, with or without menthol, can be placed directly into the cartridge or a cassette. In such an event, the cartridge or cassette will be sealed, for example by enclosing it inside an air-impervious pouch, a blister package or the like. The disadvantage of this arrangement is that it will typically be necessary to use up the entire supply of films inside the cartridge over a limited period of time to prevent their degradation over time.

Alternatively, the films placed inside the cartridge can themselves be packaged into sealed container 4 illustrated in FIG. 1, and a plurality of such containers, each holding one melatonin-carrying film, is placed inside the cartridge. When the user requires a strip, he opens cover 18, slides out one of the packages, and then opens the sealing membrane 10 on the container as above described.

The thickness of cartridge 14 shown in FIG. 2 is preferably kept as thin as possible for holding the desired number of films so that the cartridge too can be carried by the user at all times to assure its availability when and where needed.

What is claimed is:

1. A thin film for the delivery of melatonin to a person's bloodstream via a mucosal membrane comprising a thin substrate of a size to be received in the person's oral cavity and made of a low-moisture, non-tacky material which rapidly dissolves when in contact with the mucosal membrane, and from 0.3 mg to 10 mg of melatonin or a melatonin analog so that, upon dissolution of the substrate, the melatonin enters the bloodstream through the mucosal membrane of the person.

2. A thin film according to claim 1 wherein the melatonin present in the substrate is one of pure melatonin or a melatonin analog.

3. A thin film according to claim 1 including between 5% and 10% by weight of menthol or menthol analog in the substrate.

4. A thin film according to claim 1 wherein the substrate is made of vegetable ingredients only.

5. A thin film according to claim 1 wherein the substrate includes some animal by-products.

6. A thin film according to claim 1 wherein the substrate includes an artificial sweetener.

7. A thin film according to claim 1 wherein the substrate includes a flavoring substance.

8. A thin film according to claim 1 including a foil wrap exterior cover sealing the film from the environment.

9. A thin film according to claim 1 including a container having a thickness only slightly greater than a thickness of the film, and a removable cover placed over the container and sealing the film in the container from the exterior thereof.

10. A method of rapidly adding melatonin to a person's bloodstream comprising
providing a thin substrate made from a low-moisture content, non-tacky material that becomes dissolved when hydrated and that is sized so that the substrate can be placed in oral cavities of persons,
adding from 0.3 mg to 10 mg melatonin, or a melatonin analog, to the substrate,
placing the substrate including the melatonin or melatonin analog against a mucosal membrane of the person in his oral cavity, and
letting the substrate dissolve while it is in contact with the mucosal membrane to thereby liberate the melatonin or melatonin analog from the substrate so that the melatonin or melatonin analog can enter the person's bloodstream via the mucosal membrane.

11. A method according to claim 10 including
packaging the substrate including the melatonin in a flat container,
applying a sealing, readily removable membrane over the container to seal the substrate from the environment,
unobtrusively placing the sealed container into a pocket carried by the person so that the container is readily accessible to the person,
during a period when the person desires to take melatonin, removing the substrate from the container, and
thereafter placing the substrate into the person's oral cavity and contacting the substrate with a mucosal membrane in the oral cavity of the person to thereby hydrate the substrate and release the melatonin into the person's bloodstream.

12. A method according to claim 11 wherein removing the substrate comprises pulling the membrane at least partially off the container and thereafter removing the substrate from the container.
